(19) United States
(12) Patent Application Publication
(10) Pub. No.: US 2017/0281806 A1
(43) Pub. Date: Oct. 5, 2017

(54) CHELATOR-TARGETING LIGAND CONJUGATES FOR CARDIOVASCULAR IMAGING

(71) Applicant: CELL>POINT, LLC, Spring, TX (US)

(72) Inventors: F. David ROLLO, Saratoga, CA (US); Jerry L. BRYANT, Houston, TX (US)

(21) Appl. No.: 15/627,831

(22) Filed: Jun. 20, 2017

Related U.S. Application Data
(63) Continuation of application No. 12/900,200, filed on Oct. 7, 2010, now abandoned.
(60) Provisional application No. 61/250,331, filed on Oct. 9, 2009.

(51) Int. Cl. A61K 51/04

(52) U.S. Cl. CPC A61K 51/0491

(57) ABSTRACT

Disclosed are methods of imaging a site in a heart of a subject to detect cardiovascular disease that involve stressing a subject, administering to the stressed subject an effective amount of a radionuclide-labeled chelator-glucose analog conjugate, and imaging the heart of the subject by detecting a signal generated by the conjugate in the heart of the subject. Also disclosed are methods of imaging a peripheral blood vessel in a subject by using a detectable amount of a radionuclide-labeled chelator-glucose analog conjugate. Also disclosed are methods of distinguishing a false positive nuclear cardiology scan from a true positive nuclear cardiology scan, methods of diagnosing congestive heart failure or cardiac ischemia that involve imaging a subject that has been administered a radionuclide-labeled chelator-glucose analog conjugate, and methods to distinguish viable from nonviable myocardium.

This application is a continuation of U.S. patent application Ser. No. 12/900,200, filed Oct. 7, 2010, which claims priority to U.S. Provisional Patent Application No. 61/250,331, filed on Oct. 9, 2009, both of which are hereby incorporated by reference in their entirety.

### BACKGROUND OF THE INVENTION

### 1. Field of the Invention

The present invention relates generally to the fields of imaging and the diagnosis, monitoring, and management of cardiovascular disease. More particularly, the invention concerns novel methods of cardiovascular imaging and assessment of disease status using radionuclide-labeled chelator-glucose analog conjugates.

### 2. Description of Related Art

Cardiovascular disease is the leading common cause of death in men and women in the United States. It is responsible for about 40 percent of all deaths in the United States, more than all forms of cancer combined. It is the leading cause of permanent disability in the workforce.

In many instances, the first indication of heart disease is a fatal heart attack or sudden death, making it a challenge for physicians to diagnose it. Symptoms, if any, are often nonspecific. Scintigraphic procedures, such as Myocardial Perfusion Imaging (MPI) using agents such as thallium, have emerged as important diagnostic tools to assess location, severity, and prognosis in patients with coronary artery disease.

Thallium scanning, however, has limitations. For example, the frequency of positive scans in patients with acute myocardial infarction is significantly higher in patients studied within 24 hours after onset compared to those studied later (Wackers et al., 1976). Also, thallium-201 scintigraphy is insensitive to small infarcts (Niess et al., 1979). Further, variability related to exercise performance and other physiologic and technical factors greatly limits the clinical usefulness of absolute thallium clearance measurements for identifying disease in individual coronary arteries (Becker et al., 1989). While other MPI agents labeled with technetium-99m have also been developed and applied, their application has resulted in a significant increase in sensitivity but little improvement in specificity over thallium imaging (Hendel et al., 1996). Thus, there is the need for improved imaging methods with a focus on increasing the specificity for the presence of cardiovascular disease.

In contrast to cardiovascular disease, significant improvements have been made in targeted molecular imaging of tumors. Targeted molecular imaging has employed radionuclide imaging modalities. These modalities (e.g., positron emission tomography, single photon emission computed tomography) are diagnostic cross-sectional imaging techniques that map the location and concentration of radionuclide-labeled radiotracers and thus facilitate the diagnosis and assessment of tumors. Radiolabeled chelator-targeting ligand conjugates using ethylenedicysteine (EC) as a chelator are finding application in diagnosing cancer and monitoring responses to cancer treatment (See Yang et al., 2006; Zareneyrizi et al., 1999; Ilgan et al., 1998; U.S. Patent Application Pub. Nos. 20080107598, 20070248537, 20050129619, and 20040166058; and U.S. Pat. Nos. 7,582,281, 7,229,604; 7,223,380, and 7,067,111).

In view of the high prevalence of cardiovascular disease and the limitations of existing imaging methods, there is the need for improved methods of imaging cardiovascular disease that are more cost-effective, sensitive, and specific than existing imaging technologies.

### SUMMARY OF THE INVENTION

The present inventors have identified certain methods of imaging cardiovascular disease that can be applied in the diagnosis and assessment of cardiovascular disease in a subject. The disease may be a disease of the heart or a disease of a peripheral blood vessel (i.e., a blood vessel that is not within the heart). These methods have benefits over existing cardiovascular imaging modalities that include improved sensitivity and specificity, improved patient convenience, and cost reduction. The technology can, for example, be applied for assessment of cardiac viability, cardiac function, assessment of the extent and severity of cardiovascular disease as it relates to other vascular diseases (e.g., peripheral artery disease (PAD)), assessment of remote cardiac events (e.g., ischemic memory), assessment of response to therapy, and assessment of prognosis.

Accordingly, provided are methods of imaging a site in a heart of a subject to detect cardiovascular disease. In certain aspects, the method comprises detecting a signal generated by the radionuclide-labeled chelator-glucose analog conjugate in the heart of the subject, wherein a site of ischemia in the heart, if present, generates a signal that is more intense than surrounding heart tissue. In some embodiments, a radionuclide-labeled chelator-glucose analog conjugate is detected in a subject that was previously administered a radionuclide-labeled chelator-glucose analog conjugate. In other embodiments, methods include a step where the subject is administered a detectable amount of a radionuclide-labeled chelator-glucose analog conjugate. In any such embodiments, the radionuclide-labeled chelator-glucose analog may be administered at any time prior to or concurrently with the detecting of a signal generated by the radionuclide-labeled chelator-glucose analog. Some methods may be practiced on a subject that has been subjected to stress, or the methods may include a step wherein the subject is stressed prior to the detecting.

In certain embodiments, a subject is identified to be tested for cardiovascular disease. Such a subject may be any subject who is to be tested for cardiovascular disease, including any of the following: one who has or is suspected of having cardiovascular disease, one who is at risk for or suspected of being at risk for developing cardiovascular disease, one who has previously been diagnosed with or previously exhibited symptoms of a cardiovascular disease, or one who has certain characteristics wherein testing for cardiovascular disease is desirable (e.g., the subject has reached an age of increased risk, the subject has a familial history of cardiovascular disease, the subject has a genetic marker that correlates with cardiovascular disease, the subject exhibits any one of many risk factors for cardiovascular disease that are known to those of skill in the art such as high blood pressure, high cholesterol, etc.).

In some aspects, methods further comprise detecting a signal generated by a radionuclide-labeled chelator-glucose analog conjugate in a site of normal heart tissue in the subject, wherein the site of normal heart tissue generates a signal that is detectable and less intense than a signal generated by ischemic heart tissue. Because normal heart tissue generates a signal that is less intense than a signal generated by ischemic heart tissue, a site of myocardial ischemia, for example, would appear as a signal that is more intense than background signal from the heart.

Another aspect of methods disclosed herein contemplates a method of imaging a site in a heart of a subject to detect cardiovascular disease. The subject may have been previously subjected to stress and imaged using nuclear imaging to determine whether there is a region of decreased perfusion suggesting the presence of ischemia. In certain embodiments such a method comprises administering to the patient at rest a detectable amount of a radionuclide-labeled chelator-glucose analog conjugate and imaging the heart of the subject to detect a signal generated by the radionuclide-labeled chelator-glucose analog conjugate. It may then be determined if a signal generated by the radionuclide-labeled chelator-glucose analog conjugate occurs in the region of the heart that showed decreased perfusion on the MPI imaging study. If the radionuclide-labeled chelator glucose analog conjugate localizes in the region of decreased perfusion, such localization will be considered evidence of ischemia (e.g., suspected myocardial ischemia) in the heart. If the chelated agent does not localize in the region of decreased perfusion noted on the MPI study, such a result will indicate that the decreased perfusion is caused by, for example, the presence of either an artifact or scar tissue from a prior cardiac event. Such an application uses the chelated conjugate as a complement to traditional MPI imaging and may replace traditional post-stress studies as well as present an alternative to Attenuation Correction imaging, which is applied to distinguish an artifact from ischemia.

The cardiovascular disease may be any cardiovascular disease. In certain embodiments, the cardiovascular disease is a disease associated with decreased blood flow to the myocardium. Non-limiting examples include a myocardial infarction, myocardial ischemia, and congestive heart failure. Other aspects relate to the presence of ischemic tissue due to a significant decrease in the perfusion of the tissue secondary to atherosclerotic plaques, transient ischemia of vessels, or twisting of vessels, pressure in or around vessels, or other events that lead to decreased perfusion. The resulting ischemic tissue will localize the chelated conjugate, and subsequent imaging using nuclear imaging techniques can be used to the detect the presence, location, and extent of ischemia in conditions that include chronic ischemic events to the brain, transient ischemic events (TIA) to the brain, ischemic bowel events, and peripheral vascular disease.

The subject can be stressed using any method known to those of ordinary skill in the art. For example, the subject may be stressed by subjecting the subject to exercise. Alternatively, the subject may be administered a pharmacological agent that mimics the effects of stress, such as dipyridamole or adenosine.

Also provided are methods of imaging tissue nourished by a peripheral blood vessel in a subject involving detecting a signal generated by a radionuclide-labeled chelator-glucose analog conjugate in the heart of the subject. The subject may have been previously administered a detectable amount of the radionuclide-labeled chelator-glucose analog conjugate, or the method may include a step wherein the subject is administered a detectable amount of the radionuclide-labeled chelator-glucose analog conjugate. The subject may have been subjected to stress prior to the detecting, or the method may include a step wherein the subject is stressed. In certain aspects, decreased blood flow to the peripheral vessel can cause ischemia to the tissue supplied by the peripheral blood vessel. The presence of ischemia is assessed by detecting a signal generated by the radionuclide-labeled chelator-glucose analog conjugate in the tissue nourished by the peripheral blood vessel of the subject. An image may be generated to view the detected signals. The image may provide for detection of tissue having decreased flow in a peripheral blood vessel of the subject.

Other methods concern distinguishing a false positive nuclear cardiology scan from a true positive nuclear cardiology scan. In such methods, the subject may be administered a detectable amount of a radionuclide-labeled chelator-glucose analog conjugate. In certain aspects, the subject has had a positive nuclear cardiology scan, and a site of diminished signal was identified on the nuclear cardiology scan that is suggestive of cardiac ischemia. The heart of the subject may then be imaged to detect the presence of a signal generated by the radionuclide-labeled chelator-glucose analog conjugate. In certain embodiments, the presence of a signal that is more intense than surrounding heart tissue is indicative of a true positive nuclear cardiology scan, and the absence of a signal that is more intense than surrounding heart tissue is indicative of a false positive nuclear cardiology scan. In methods that include a step of administering a radionuclide-labeled chelator-glucose analog, the radionuclide-labeled chelator-glucose analog conjugate may be administered concurrently with the another agent, such as radiolabeled MPI agent, prior to the nuclear cardiology scan. Imaging of the radio-labeled chelator-glucose analog conjugate may occur from about 5 to about 180 minutes following injection of the agent into the subject. In other methods, including methods where the subject undergoing examination has already been administered a radionuclide-labeled chelator-glucose analog conjugate and imaged to assess the presence of ischemia, a repeat study can be performed up to about 8 weeks following the baseline imaging study to determine whether ischemia persists and to determine whether the extent of the ischemia has decreased as a result of revascularization therapy or medical management. In some embodiments the radionuclide-labeled chelator-glucose analog conjugate imaging study may be performed up to about 8 weeks following the baseline positive nuclear cardiology scan. In some embodiments, repeat imaging is performed within 5 minutes to 8 weeks following the baseline nuclear cardiology scan. In other embodiments, repeat imaging is performed within 5 minutes to 4 weeks following the baseline nuclear cardiology scan. In further embodiments, repeat imaging is performed within 5 minutes to 1 week following the baseline nuclear cardiology scan. In still further embodiments, imaging is performed within 5 minutes to 1 day following the baseline nuclear cardiology scan. In more particular embodiments, imaging is performed within 5 minutes to one hour following the baseline nuclear cardiology scan. In certain aspects, imaging is performed 1, 2, 3, 4, 5, 6, 7, 8, 9 or 10 days or weeks following the baseline nuclear cardiology scan. In some aspects, imaging is performed 5, 10, 15, 20, 25, 30, 35, 40, 45, 50, or 55 minutes or hours following the baseline nuclear cardiology scan.

In some embodiments, the nuclear cardiology scan (MPI) of the heart is repeated. In particular embodiments, the nuclear cardiology scan involves thallium scintigraphy. Non-limiting examples of imaging agents administered for performance of the nuclear cardiology scan include technetium Tc-99m tetrofosmin (Myoview™) or Technetium Tc99m Sestamibi (Cardiolite®). In some embodiments, thallium-201, technetium Tc-99m tetrofosmin (Myoview™), or Technetium Tc99m Sustamibi (Cardiolite®) is administered concurrently with the radionuclide-labeled chelator-glucose analog prior to or during imaging.

Imaging or detecting may be performed using the same camera or different cameras, and in part depends on the particular imaging agent employed for the nuclear cardiology scan.

Some other aspects concern methods to diagnose congestive heart failure or monitor responses to treatment of congestive heart failure in a subject. In certain embodiments, such a method comprises detecting a signal generated by a radionuclide-labeled chelator-glucose analog conjugate in the heart of the subject, wherein a site of ischemia in the heart, if present, generates a signal that is more intense than surrounding heart tissue and is indicative of the presence of cardiac ischemia. In some methods, a subject is identified to be tested for congestive heart failure or to be monitored in conjunction with treatment of congestive heart failure. Some methods further include administering to the subject a detectable amount of a radionuclide-labeled chelator-glucose analog conjugate. Some methods may also include performing one or more medical studies selected from the group consisting of a chest X-ray, a CT scan of the heart, an Mill scan of the heart, an echocardiogram, and an angiographic procedure. In some embodiments, the subject has a history of having undergone a nuclear cardiology study.

Further embodiments concern methods to diagnose a previous episode of cardiac ischemia in a subject that is suspected of having had a previous episode of cardiac ischemia. In certain embodiments, the method includes detecting a signal generated by a radionuclide-labeled chelator-glucose analog conjugate in the heart of the subject, wherein a site of ischemia in the heart, if present, generates a signal that is more intense than surrounding heart tissue and is indicative of the presence of past or current cardiac ischemia. Some methods include a step of identifying a subject that is suspected of having had a previous episode of cardiac ischemia. The previous episode of cardiac ischemia may have been symptomatic or asymptomatic. The subject may be a subject that has undergone a previous nuclear cardiology study. The previous nuclear cardiology study may have been a study suggestive of or negative for a site of cardiac ischemia. In some embodiments, the subject has a history of an episode of chest pain or other symptom attributable to the heart that has since resolved, and imaging is performed within about one week to within about eight weeks following the episode of chest pain. In other embodiments, the subject has a history of an episode of chest pain or other symptom attributable to the heart that has since resolved, and imaging is performed within two weeks following the episode of chest pain. In further embodiments, the subject has a history of an episode of chest pain or other symptom attributable to the heart that has since resolved, and imaging is performed within four weeks following the episode of chest pain. In still further embodiments, the subject has a history of an episode of chest pain or other symptom attributable to the heart that has since resolved, and imaging is performed within eight weeks following the episode of chest pain. In particular embodiments, the subject has a history of an episode of chest pain or other symptom attributable to the heart that has since resolved, and imaging is performed within two weeks to eight weeks following the episode of chest pain. Some embodiments further including repeating the imaging. In some aspects, an image is generated from the detected signals. The imaging study may be performed without the use of a stress test or using pharmacologic stress agents. In further aspects, the subject may be stressed or administered a pharmacologic stress agent prior to the detecting.

The subject may be any mammalian subject. Non-limiting examples include a human, a primate, a horse, a cow, a pig, a goat, a sheep, a rabbit, a dog, a cat, a rat, and a mouse. In particular embodiments, the subject is a human. The human may be a patient with known or suspected cardiovascular disease or any patient for which testing for a cardiovascular disease is desirable.

The chelator may be any chelator known to those of ordinary skill in the art that is capable of chelating to a radionuclide. In some embodiments, the chelator is of Formula I:

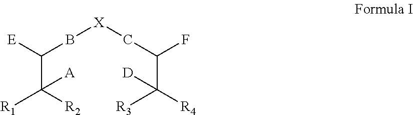

wherein the point of conjugation between the chelator and the glucose analog is at one or more positions selected from the group consisting of A, B, C, D, E and F; A, D, E and F are each independently H, lower alkyl, —COOH, —NH₂, or thiol; B and C are each independently a secondary amine, a tertiary amine, —S—, —S(O)—, or —S(O)₂—; R₁, R₂, R₃ and R₄ are each independently H or lower alkyl; and X is selected from the group consisting of —CH₂—CH₂—, —CH₂—CH₂—CH₂—, —CH₂—C(O)—, —C(O)—CH₂—, —C(O)—CH₂—CH₂— and —CH₂—CH₂—C(O)—. In some embodiments, any three or four of the groups A, B, C, D, E and F together form a chelate selected from the group consisting of NS₂, N₂S, S₄, N₂S₂, N₃S and NS₃. Any one of at least A, D, E, and F may optionally be a thiol. In particular embodiments, the chelate is N₂S₂. In some embodiments, at least one of A, D, E and F may include a primary amine or at least one of B and C may include a secondary amine. In particular embodiments, E and F are each independently selected from the group consisting of —COOH, —NH₂ or thiol. In some embodiments, the conjugation of at least one targeting ligand takes place at E and/or F. In a particular embodiment, the chelator is ethylenedicysteine (EC).

The glucose analog may be any analog of glucose known to those of ordinary skill in the art. “Glucose analog” as used herein refers to a chemical compound that is structurally or functionally similar to glucose. In some embodiments, the glucose analog is a compound of Formula II:

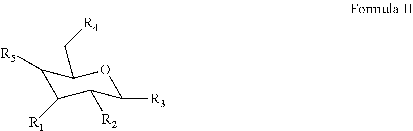

wherein R1, R2, R3, R4, R5, and R6 are each separately selected from the group consisting of H, hydroxy, amino, thiol, halogen, alkyl, alkoxy, substituted alkyl, phosphate, an amino acid, or a consecutive series of more than one amino acids.

In some particular embodiments, the glucose analog is glucosamine or deoxyglucose. In other embodiments, the glucose analog is an aminoglycoside. Non-limiting examples of aminoglycosides include neomycin, kanamycin, gentamycin, amikacin, tobramycin, netilmicin, ribostamycin, sisomicin, micromicin, lividomycin, dibekacin, isepamicin, and astromicin. In particular embodiments, the chelator-glucose analog is EC-glucosamine or EC-deoxyglucose.

The chelator-glucose analog conjugate may optionally include a linker between the chelator and the glucose analog. Non-limiting examples of linkers include a peptide, glutamic acid, aspartic acid, bromo ethylacetate, ethylene diamine, lysine and any combination of one or more of these groups.

The radionuclide may be any radionuclide known to those of ordinary skill in the art. Non-limiting examples of radionuclides include ^{99m}Tc, ¹⁸⁸Re, ¹⁸⁷Re, ¹⁸⁶Re, ¹⁵³Sm, ¹⁶⁶Ho, ⁹⁰Y, ⁸⁹Sr, ⁶⁷Ga, ⁶⁸Ga, ¹¹¹In, ¹⁸³Gd, ⁵⁹Fe, ²²⁵Ac, ²¹²Bi, ²¹¹At, ⁴⁵Ti, ⁶⁰Cu, ⁶¹Cu, ⁶⁷Cu, ⁶⁴Cu and ⁶²Cu. In particular embodiments, the radionuclide is ^{99m}Tc. In more particular embodiments, the radionuclide is ^{99m}Tc (oxo). In particular embodiments, the radionuclide-labeled chelator-glucose analog is ^{99m}Tc (oxo)-labeled EC-glucosamine or ^{99m}Tc (oxo)-labeled EC-deoxyglucose.

In some embodiments, the radionuclide-labeled chelator-targeting ligand conjugate is of Formula III:

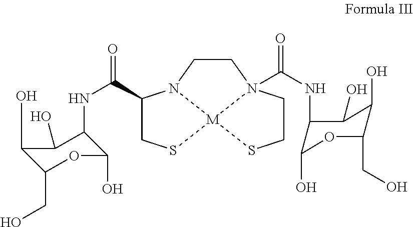

wherein M is a radionuclide.

In further embodiments, the radionuclide-labeled chelator-targeting ligand conjugate is of Formula IV:

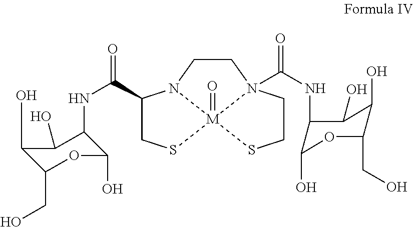

wherein M is a radionuclide capable of forming a double bond with an oxygen atom. In a particular embodiment, M is ^{99m}Tc.

The chelator-glucose analog conjugate may optionally be conjugated to one or more other ligands. Non-limiting examples of other ligands include a cardiovascular drug, a cardiac ischemia marker, a cardiac viability tissue marker, a congestive heart failure marker, a rest/stress cardiac tissue marker, and an imaging moiety. Non-limiting examples of cardiovascular drugs include an antihyperlipoproteinemic agent, an antiarteriosclerotic agent, an antithrombotic agent, a fibrinolytic agent, an antiplatelet agent, a blood coagulant, a thrombolytic agent, an antiarrythmic agent, an antihypertensive agent, a vasopressor, an anti-angiotension II agent, an afterload-preload reduction agent, a diuretic, and an inotropic agent. Non-limiting examples of cardiac ischemia markers include interleukin-6, tumor necrosis factor alpha, matrix metalloproteinase 9, myeloperoxidase, an intercellular adhesion molecule, a vascular adhesion molecule, soluble CD40 ligand, placenta growth factor, high sensitivity C-reactive protein, ischemia modified albumin, a free fatty acid, choline, and adenosine. Non-limiting examples of cardiac viable tissue markers include phospholipase C, myosin light-chain phosphatase, nitric oxide, prostacyclin, endothelin, thromboxane, L-arginine and L-citrulline. Non-limiting examples of congestive heart failure markers include interleukin-1, cardiotrophin-1, insulin-like growth factor, epidermal growth factor, tyrosine kinase receptor, angiotensin II, and metronidazole. Non-limiting examples of rest/stress cardiac tissue markers include a mitogen-activated protein kinase, cyclic adenosine monophosphate, phospholipase C, phosphatidylinositol bisphosphate, isositol trisphosphate, diacylglycerol, a tyrosine kinase, and metronidazole.

The radionuclide-labeled chelator-glucose analog conjugate may be administered to a subject in a pharmaceutical composition that includes one or more additional agents. In some embodiments, the composition further includes a reducing agent. Non-limiting examples of reducing agents include a dithionite ion, a stannous ion and a ferrous ion.

Detecting or imaging can be performed using any method known to those of ordinary skill in the art. In particular embodiments, detecting or imaging is performed using SPECT imaging, or SPECT imaging in combination with CT imaging. Other embodiments include SPECT imaging devices equipped with a radionuclide or x-ray source for Attenuation Correction. Other imaging modalities include PET imaging or PET imaging in combination with another type of imaging, such as CT imaging. In a particular embodiment, PET imaging is used in combination with CT imaging equipped with a radionuclide or x-ray source for Attenuation Correction.

In some embodiments, the subject is further administered a second agent for nuclear imaging of the heart. The second agent for nuclear imaging of the heart can be any such agent known to those of ordinary skill in the art. For example, the second agent may be selected from the group consisting of radioactive thallium-201, technetium Tc-99m tetrofosmin (Myoview™), and Tc-99m Sestamibi)(Cardiolite®). The second agent may be administered before, concurrently with, or following administration of the radionuclide-labeled chelator-glucose analog.

A first image may be obtained following administration of the radionuclide-labeled chelator-glucose analog, and a second image may be obtained following administration of the second agent. The first image may be obtained prior to, concurrently with, or following the second image. The same camera or different cameras may be used to obtain the first image and the second image. Some embodiments of the invention involve comparing the first image to the second image. Some embodiments involving taking a single image following administration of the radionuclide-labeled chelator-glucose analog and the second agent. When taking a single image following administration of the radoionuclide-labeled chelator-glucose analog and a second agent, whether it be an agent labeled with the same Tc-99m as used for the chelator-glucose analog or a different radioisotope such as Thallium-201, Indium-111 or other acceptable labeling agents, the imaging process is referred to in the standard practice of nuclear imaging as Simultaneous Dual Isotope (SDI) imaging or Dual Isotope Imaging (DI) and is a method to display a single image for the interpreting physician which differentiates one radiolabeled agent from the second by color coding or subtracting one image from the other resulting in the localization of the individual agents.

Detecting or imaging may be performed at any time point after a subject has been administered a radionuclide-labeled chelator-glucose analog. In some embodiments, dynamic imaging is performed to determine the uptake characteristics of the agent in the normal as well as ischemic myocardium. For example, the imaging may be performed concurrently with administration of the radionuclide-labeled chelator-glucose analog. In other embodiments, imaging is performed within 30 minutes following administration of the radionuclide-labeled chelator-glucose analog. I some aspects, imaging may be performed up to 3 hours following administration of the radionuclide-labeled chelator-glucose analog conjugate. In aspects involving repeat imaging, the repeat imaging may be performed within about 2 days to within about 7 days of a prior or baseline study. In certain aspects, repeat imaging may be performed within about 2 weeks to within about 8 weeks of the prior or baseline study. Any repeat imaging may be dynamic imaging or imaging performed within about 30 minutes to within about 3 hours following administration of the radionuclide-labeled agent. In some embodiments, repeat imaging following administration of the radionuclide-labeled chelator-glucose analog is performed between two weeks and eight weeks after the baseline study.

The radionuclide-labeled chelator-glucose analog conjugate may be administered at any diagnostically detectable dose contemplated by one of ordinary skill in the art. For example, the dose may be a dose of about 5 mCi to about 100 mCi, a dose of about 10 mCi to about 50 mCi, or a dose of about 20 mCi to about 40 mCi. In particular embodiments, the dose that is administered is about 5mCi to about 10 mCi. In other embodiments, the dose that is administered is about 30 mCi or about 40mCi. In additional embodiments, the dose that is administered is about 2, 3, 4, 5, 6, 7, 8, 9, 10, 11, 12, 13, 14, 15, 16, 17, 18, 19, or 20 mCi.

It is contemplated that any embodiment discussed in this specification can be implemented with respect to any method, compound or composition of the invention, and vice versa. Furthermore, compounds and compositions of the invention can be used to achieve methods of the invention.

A person of ordinary skill in the art will recognize that chemical modifications can be made to the compounds of the present invention, as well as compounds employed in the method of the present invention, without departing from the spirit and scope of the present invention. Substitutes, derivatives, or equivalents can also be used, all of which are contemplated as being part of the present invention.

The word “conjugate” and “conjugated” is defined herein as chemically joining within the same molecule. For example, two or more molecules and/or atoms may be conjugated together via a covalent bond, forming a single molecule. The two molecules may be conjugated to each other via a direct connection (e.g., where the compounds are directly attached via a covalent bond) or the compounds may be conjugated via an indirect connection (e.g., where the two compounds are covalently bonded to one or more linkers, forming a single molecule). In other instances, a metal atom may be conjugated to a molecule via a chelation interaction.

As used herein, “chelate” may be used as a noun or a verb. As a noun, “chelate” (or “chelator”) refers to a compound having one or more atoms that are either capable of chelating one or more metal ions, or are chelating to one or more metal ions. In preferred embodiments, only one metal ion coordinates to a chelate. A non-limiting example of “chelate” includes “an N₂S₂” chelate: this means that two nitrogen atoms and two sulfur atoms of a chelator are either a) capable of chelating to one or more metal ions or b) are coordinated to (or chelated to) to one or more metal ions (preferably just one metal ion). As a verb, “chelate” refers to the process of a metal ion becoming coordinated or chelated to, for example, a chelator or a chelator-targeting ligand conjugate.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects. For example, “about” can be within 10%, preferably within 5%, more preferably within 1%, and most preferably within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

Any embodiment of any of the present methods may consist of or consist essentially of—rather than comprise/include/contain/have—the described limitations and/or steps. Thus, in any of the claims, the term “consisting of” or “consisting essentially of” may be substituted for open-ended language in a given claim in order to change the scope of the claim from what it would otherwise be using the open-ended linking verb.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Comparison of uptake of T1-201 and 99mTc-EC-glucosamine in heart tissue of dogs as measured by autoradiography. The bar graph indicates that there was significant enhancement of the uptake of 99mTc-EC-glucosamine in low flow tissue compared to the uptake of T1-201 in the same tissue.

FIG. 2. Time line of protocol concerning ischemic memory. LAD flow was reduced by 75% tightening of a snare occluder in dogs to produce resting ischemia for 90 minutes followed by full perfusion. 99mTc-EC-glucosamine was injected 15 minutes after reflow with SPECT performed at one hour post injection.

FIG. 3. Results of LAD/LCx flow in two dogs following placement of a snare occluder to reduce LAD flow by 75%. After reperfusion, both flow and T1-201 activity in the LAD zone were slightly reduced relative to the normal zone, but 99mTc-EC-glucosamine activity in the LAD zone was significantly higher than in the normal LCx zone.

FIG. 4. 99mTc-EC-glucosamine uptake (ECDG uptake) vs. stenotic flow in the acute low flow model.

### DESCRIPTION OF ILLUSTRATIVE EMBODIMENTS

The present inventors have found that radionuclide-labeled chelator-glucose analog conjugates have broad application in the evaluation of patients with a known or suspected cardiovascular disease. Imaging methods employing these conjugates can identify sites of cardiac ischemia, thus resulting in improved methods of evaluation of subjects with known or suspected cardiovascular disease.

### A. Chelators

The present methods concern applications involving radiolabeled chelator-glucose analog conjugates. Persons of skill in the art will be familiar with compounds capable of chelating one or more metal ions (“chelators”). The chelators employed in the methods of the present invention generally comprise one or more atoms capable of chelating to one or more radionuclide ions. Chelators comprising three or four atoms available for chelation are preferred. Typically, a chelator chelates to one radionuclide.

Chelation of a radionuclide to a chelator can be by any method known to those of ordinary skill in the art. Atoms available for chelation typically comprise O, N or S. In certain particular embodiments, the metal ion is chelated to a group of atoms, referred to herein as “chelates,” selected from the group consisting of NS₂, N₂S, S₄, N₂S₂, N₃S and NS₃. Chelation can also occur among both the chelator and the targeting ligand—i.e., both the chelator and the targeting ligand may contribute atoms that chelate the same metal ion.

In some embodiments, the chelator is a compound that includes one or more amino acid moieties. For example, the amino acid may be cysteine or glycine. A spacer may connect one amino acid to another.

It is well known to those of ordinary skill in the art that chelators, in general, comprise a variety of functional groups. Non-limiting examples of such functional groups include hydroxy, thiol, amine, amido and carboxylic acid.

One example of a chelator is a compound of Formula I:

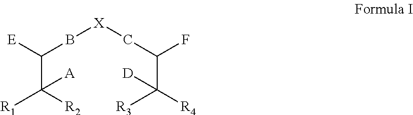

wherein:
A, D, E and F are each independently H, lower alkyl, —COOH, —NH₂, or thiol;
B and C are each independently a secondary amine, a tertiary amine, —S—, —S(O)—, or —S(O)₂—;
R₁, R₂, R₃ and R₄ are each independently H or lower alkyl; and
X is selected from the group consisting of —CH₂—CH₂—, —CH₂—CH₂—CH₂—, —CH₂—C(O)—, —C(O)—CH₂—, —C(O)—CH₂—CH₂— and —CH₂—CH₂—C(O)—.

In some embodiments, the chelator is a molecule that includes one or more carboxyl (—COOH) moieties. In some embodiments, the chelator is a molecule that includes one or more amino (—NH₂) moieties. In some embodiments, the chelator is a molecule that includes two or more thiol groups.

In some embodiments, the chelator is a bis-aminoethanethiol (BAT) dicarboxylic acid. A non-limiting example of a bis-aminoethanethiol dicarboxylic acid is ethylenedicysteine (EC). BAT dicarboxylic acids are capable of acting as tetradentate ligands, and are also known as diaminodithiol (DADT) compounds. These compounds are known to form stable Tc(V)O-complexes on the basis of efficient binding of the oxotechnetium group to two thiol-sulfur and two amine-nitrogen atoms.

Chelators contemplated for use in the present methods may comprise one or more spacers. Such spacers are well known to those of ordinary skill in the art. These spacers, in general, provide additional flexibility to the overall compound that may facilitate chelation of one or more metal ions to the chelator. Non-limiting examples of spacers include alkyl groups of any length, such as ethylene (—CH₂—CH₂—), ether linkages, thioether linkages, amine linkages, and any combination of one or more of these groups. In some embodiments, multiple chelators linked together are capable of forming a molecule that may chelate to one or more radionuclide ions.

### B. Radionuclides

As set forth above, conjugates labeled with one or more radionuclides are contemplated for use in methods of the present invention. A radionuclide is an isotope of artificial or natural origin that exhibits radioactivity. In some embodiments, the radionuclide is selected from the group consisting of ^{99m}Tc, ¹⁸⁸Re, ¹⁸⁶Re, ¹⁵³Sm, ¹⁶⁶Ho, ⁹⁰Y, ⁸⁹Sr, ⁶⁷Ga, ⁶⁸Ga, ¹¹¹In, ¹⁴⁸Gd, ⁵⁵Fe, ²²⁵Ac, ²¹²Bi, ²¹¹At, ⁴⁵Ti, ⁶⁰Cu, ⁶¹Cu, ⁶⁷Cu, and ⁶⁴Cu. In particular embodiments, the metal ion is rhenium or a radionuclide such as ^{99m}Tc, ¹⁸⁸Re, or ⁶⁸Ga. In particular embodiments, oxo[99mTc]technetium (V) is bound to the chelator-glucose analog conjugate employed herein.

As described below, a reducing agent may need to accompany one of the radionuclides, such as ^{99m}Tc. Non-limiting examples of such reducing agents include a dithionite ion, a stannous ion and a ferrous ion.

A number of factors must be considered for optimal radioimaging in humans. To maximize the efficiency of detection, a metal ion that emits gamma energy in the 100 to 200 keV range is preferred. A “gamma emitter” is herein defined as an agent that emits gamma energy of any range. One of ordinary skill in the art would be familiar with the various metal ions that are gamma emitters. To minimize the absorbed radiation dose to the patient, the physical half-life of the radionuclide should be as short as the imaging procedure will allow. To allow for examinations to be performed on any day and at any time of the day, it is advantageous to have a source of the radionuclide always available at the clinical site. ^{99m}Tc is a preferred radionuclide because it emits gamma radiation at 140 keV, it has a physical half-life of 6 hours, and it is readily available on-site using a molybdenum-99/technetium-99m generator. One of ordinary skill in the art would be familiar with methods to determine optimal radioimaging in humans.

### C. Glucose Analogs

Conjugates employed in the methods of the present invention include a glucose analog. A “glucose analog” as defined herein refers to a compound that has structural similarity to a glucose molecule. A glucose analog may also be a glucose derivative, which includes any molecule derived from glucose. Non-limiting examples of glucose derivatives include deoxyglucose, 2-Deoxy-D-Glucose, alpha-D-glucopyranose, beta-D-glucopyranose, 3-phospho-D-glycerate, alpha-D-glucose-1-phosphate, alpha-D-glucose-6-phosphate, beta-D-glucose-6-phosphate, beta-D-glucuronate, beta-D-glucosamine, beta-D-glucosamine-6-phosphate, D-glucosamine-6-phosphate, D-glucosamine, D-glucosaminide, D-glucosaminyl-D-glucosaminide, glucose-1,6-bisphosphate, glucose-1-phosphate, glucose-6-phosphate, and others known to those in the art. The glucose analog may also be a glycoprotein.

In some embodiments, the glucose analog is a compound of Formula II:

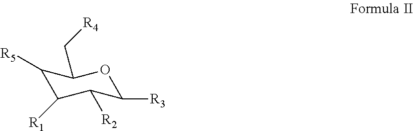

wherein R1, R2, R3, R4, R5, and R6 are each separately selected from the group consisting of H, hydroxy, amino, thiol, halogen, alkyl, alkoxy, substituted alkyl, phosphate, an amino acid, or a consecutive series of more than one amino acids.

The term “hydroxy” as used herein refers to —OH or —O⁻.

The term “amino” as used herein refers to the group —NRR′, where R and R′ may independently be hydrogen, alkyl, aryl, alkoxy, or heteroaryl. The term “aminoalkyl” as used herein represents a more detailed selection as compared to “amino” and refers to the group —NRR′, where R and R′ may independently be hydrogen or (C₁-C₄)alkyl.

“Amino acid” refers to a naturally occurring amino acid. “Consecutive series of one or more amino acids” refers to a consecutive series of naturally occurring amino acids.

“Halogen” refers to any halogen atom known to those of ordinary skill in the art. For example, the halogen may be fluorine, chlorine, bromine, or iodine. The halogen atom may be a radioactive halogen. Non-limiting examples of radioactive halogens include 18F, 121I, 123I, 124I, 125I, 131I, 75Br, 76Br, 77Br, and 34Cl.

As used herein, “alkyl” refers to a straight, branched or cyclic carbon-carbon or hydrocarbon chain, optionally including alkene or alkyne bonding, containing 1-30 carbons. Non-limiting examples of alkyls include methyl, ethyl, propyl, butyl and isopropyl.

As used herein, the term “alkoxy” includes substituted and unsubstituted alkyl, alkenyl, and alkynyl groups covalently linked to an oxygen atom. In one embodiment, the alkoxy group contains 1 to 4 carbon atoms. Embodiments of alkoxy groups include, but are not limited to, methoxy, ethoxy, isopropyloxy, propoxy, butoxy, and pentoxy groups. Embodiments of substituted alkoxy groups include halogenated alkoxy groups. In a further embodiment, the alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonyl amino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties.

The term “phosphate” as used herein refers to a moiety of Formula V:

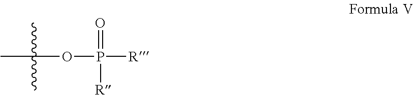

wherein R″ and R′″ are each independently selected from the group consisting of —OH, —O⁻, or alkyl.

Compounds as described herein may contain one or more asymmetric centers and thus can occur as racemates and racemic mixtures, single enantiomers, diastereomeric mixtures and individual diasteromers. All possible stereoisomers of the all the compounds described herein, unless otherwise noted, are contemplated as being within the scope of the present invention. The chiral centers of the compounds of the present invention can have the S- or the R-configuration, as defined by the IUPAC 1974 Recommendations. The present invention is meant to comprehend all such isomeric forms of the compounds of the invention.

Other examples of glucose analogs include aminoglycosides. Non-limiting examples of aminoglycosides include neomycin, kanamycin, gentamycin, amikacin, tobramycin, netilmicin, ribostamycin, sisomicin, micromicin, lividomycin, dibekacin, isepamicin, and astromicin.

In terms of structure, agents that mimic glucose typically have a glucose ring structure. Exceptions exist, however, such as puromycin, which has a pentose ring structure, but which can still be considered an agent that mimics glucose.

In terms of function, aminoglycosides are used as antibiotics that block the glycolysis pathway by their property of being structurally similar to glucose and thus, they are functionally considered as agents that mimic glucose. When these aminoglycosides are used in imaging studies, there are no detectable pharmacological effects.

Non-limiting examples of chemical structures with their PubChem Database (NCBI) identifier CID number are as follows: Amikacin CID 37768; Aminoglycoside CID 191574; Astromicin CID 65345; Deoxy-glucose CID 439268; D-glucosamine CID 441477; Dibekacin CID 3021; Gentamicin CID 3467; Glucose CID 5793; Isepamicin CID 456297; Kanamycin CID 5460349; Lividomycin CID 72394; Micromicin CID 107677; Neomycin CID 504578; Netilmycin CID 441306; Puromycin CID 439530; Ribostamycin CID 33042; Sisomicin CID 36119; and Tobramycin CID 36294.

References which describe the glycolysis blocking by aminoglycosides include, for example, Tachibana et al., 1976; Borodina et al., 2005; Murakami et al., 1996; Hoelscher et al., 2000; Yang et al., 2004; Michalik et al., 1989; Murakami et al., 1997; Diamond et al., 1978; Hostetler and Hall, 1982; Benveniste and Davies, 1973; Hu, 1998; Yanai et al., 2006; Myszka et al., 2003; Nakae and Nakae, 1982; Ozmen et al., 2005; and Tod et al., 2000.

### D. Other Ligands

In some embodiments, the radionuclide-labeled chelator-glucose analog targeting ligand includes one or more additional other ligands. Non-limiting examples of such other ligands include therapeutic ligands, ligands that can target diseased cardiovascular tissue, and imaging moieties. These ligands can be conjugated using any method known to those of ordinary skill in the art. For example, the ligand may be conjugated via a functional moiety. Non-limiting examples of functional groups include carbon-carbon bonds (including single, double and triple bonds), hydroxyl (or alcohol), amine, sulfhydryl (or thiol), amide, ether, ester, thioether, thioester, carboxylic acid and carbonyl groups. As used herein, “amine” and “amino” and other similar pairs of words such as “hydroxy” and “hydroxyl” refer to the same functional moiety and thus are used interchangeably. As used herein, “amine” may refer to either or both —NH₂ and —NH—.

Information pertaining to conjugation of ligands to chelators is provided in U.S. Pat. Nos. 6,692,724, 7,223,380, 7,582,281; U.S. patent application Ser. Nos. 09/599,152, 10/627,763, 10/672,142, 10/703,405, 10/732,919, 11/405,334, and 11/770,395, each of which is herein specifically incorporated by reference in its entirety for this section of the specification and all other sections of the specification.

Representative examples of other ligands are discussed below.

1. Cardiovascular Drugs

A “cardiovascular drug” refers to any therapeutic agent that can be applied in the treatment or prevention of a disease of the heart and/or blood vessels. Examples of such drugs are set forth in U.S. patent application Ser. No. 11/770,395, herein specifically incorporated by reference.

In certain embodiments, the cardiovascular drug is an agent that lowers the concentration of one of more blood lipids and/or lipoproteins, known herein as an “antihyperlipoproteinemic,” which can be applied in the treatment of athersclerosis and thickenings or blockages of vascular tissues. Examples include an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof. Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate. Non-limiting examples of resins/bile acid sequesterants include cholestyramine (cholybar, questran), colestipol (colestid) and polidexide. Non-limiting examples of HMG CoA reductase inhibitors include lovastatin (mevacor), pravastatin (pravochol) or simvastatin (zocor). Non-limiting examples of nicotinic acid derivatives include nicotinate, acepimox, niceritrol, nicoclonate, nicomol and oxiniacic acid. Non-limiting examples of thyroid hormones and analogs thereof include etoroxate, thyropropic acid and thyroxine. Non-limiting examples of miscellaneous antihyperlipoproteinemics include acifran, azacosterol, benfluorex, β-benzalbutyramide, carnitine, chondroitin sulfate, clomestrone, detaxtran, dextran sulfate sodium, 5, 8, 11, 14, 17-eicosapentaenoic acid, eritadenine, furazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

Non-limiting examples of an antiarteriosclerotic include pyridinol carbamate.

In certain embodiments, the cardiovascular drug is an agent that aids in the removal or prevention of blood clots. Non-limiting examples of antithrombotic and/or fibrinolytic agents include anticoagulants, anticoagulant antagonists, antiplatelet agents, thrombolytic agents, thrombolytic agent antagonists or combinations thereof. Examples of antithrombotic agents include aspirin and warfarin (coumadin). Examples of anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin. Non-limiting examples of antiplatelet agents include aspirin, a dextran, dipyridamole (persantin), heparin, sulfinpyranone (anturane) and ticlopidine (ticlid). Non-limiting examples of thrombolytic agents include tissue plaminogen activator (activase), plasmin, pro-urokinase, urokinase (abbokinase) streptokinase (streptase), anistreplase/APSAC (eminase).

In some embodiments, the cardiovascular drug is a blood coagulant. Non-limiting examples of a blood coagulation promoting agent include thrombolytic agent antagonists and anticoagulant antagonists. Non-limiting examples of anticoagulant antagonists include protamine and vitamine K1.

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

The cardiovascular drug may be an antiarrythmic agent. Non-limiting examples of antiarrhythmic agents include Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents. Non-limiting examples of sodium channel blockers include Class IA, Class D3 and Class IC antiarrhythmic agents. Non-limiting examples of Class IA antiarrhythmic agents include disppyramide (norpace), procainamide (pronestyl) and quinidine (quinidex). Non-limiting examples of Class D3 antiarrhythmic agents include lidocaine (xylocaine), tocainide (tonocard) and mexiletine (mexitil). Non-limiting examples of Class IC antiarrhythmic agents include encainide (enkaid) and flecainide (tambocor). Non-limiting examples of a beta blocker, otherwise known as a β-adrenergic blocker, a β-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol. Non-limiting examples of an agent that prolong repolarization, also known as a Class III antiarrhythmic agent, include amiodarone (cordarone) and sotalol (betapace). Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (nifedipine-type) calcium antagonist. Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

Other examples of cardiovascular drugs include antihypertensive agents. Non-limiting examples of antihypertensive agents include sympatholytic, alpha/beta blockers, alpha blockers, anti-angiotensin II agents, beta blockers, calcium channel blockers, vasodilators and miscellaneous antihypertensives. Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin. In certain embodiments, an antihypertensive agent is both an alpha and beta adrenergic antagonist. Non-limiting examples of an alpha/beta blocker comprise labetalol (normodyne, trandate). Non-limiting examples of anti-angiotension II agents include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan. Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a B-adrenergic blocking agent or a alpha1-adrenergic blocking agent. Non-limiting examples of a ganglion blocking agent include mecamylamine (inversine) and trimethaphan (arfonad). Non-limiting examples of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin). In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimefylline, trapidil, tricromyl, trimetazidine, trolnitrate phosphate and visnadine. In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative. Non-limiting examples of arylethanolamine derivatives include amosulalol, bufuralol, dilevalol, labetalol, pronethalol, sotalol and sulfinalol. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide. Non-limiting examples of N-carboxyalkyl(peptide/lactam) derivatives include alacepril, captopril, cilazapril, delapril, enalapril, enalaprilat, fosinopril, lisinopril, moveltipril, perindopril, Non-limiting examples of dihydropyridine derivatives include amlodipine, felodipine, isradipine, nicardipine, nifedipine, nilvadipine, nisoldipine and nitrendipine. Non-limiting examples of guanidine derivatives include bethanidine, debrisoquin, guanabenz, guanacline, guanadrel, guanazodine, guanethidine, guanfacine, guanochlor, guanoxabenz and guanoxan. Non-limiting examples of hydrazines/phthalazines include budralazine, cadralazine, dihydralazine, endralazine, hydracarbazine, hydralazine, pheniprazine, pildralazine and todralazine. Non-limiting examples of imidazole derivatives include clonidine, lofexidine, phentolamine, tiamenidine and tolonidine. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate. Non-limiting examples of reserpine derivatives include bietaserpine, deserpidine, rescinnamine, reserpine and syrosingopine. Non-limiting examples of sulfonamide derivatives include ambuside, clopamide, furosemide, indapamide, quinethazone, tripamide and xipamide.

Other examples of cardiovascular drugs include vasopressors. Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

Other examples of cardiovascular drugs include agents that can be applied in the treatment or prevention of congestive heart failure. Non-limiting examples of agents for the treatment of congestive heart failure include anti-angiotension II agents, afterload-preload reduction treatment, diuretics and inotropic agents. Examples of afterload-preload reduction agents include hydralazine (apresoline) and isosorbide dinitrate (isordil, sorbitrate). Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-di sulfonamide, di sulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexiline, ticrnafen and urea. Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol. In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor). Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

2. Angiogenesis Targeting Ligands

The radionuclide-labeled chelator-glucose analog conjugates set forth herein may further be conjugated to an angiogenesis targeting ligand. Angiogenesis targeting ligands are discussed in U.S. patent application Ser. No. 11/770,395, herein incorporated by reference in its entirety. “Angiogenesis targeting ligands” refers to agents that can bind to neovascularization or revascularization of tissue. For example, the neovascularization of tumor cells or revascularization of myocardium tissue. Agents that are used for this purpose are known to those of ordinary skill in the art for use in performing various measurements, including measurement of the size of a tumor vascular bed and measurement of tumor volume. Some of these agents bind to the vascular wall. One of ordinary skill in the art would be familiar with the agents that are available for use for this purpose.

Throughout this application, “angiogenesis targeting” refers to the use of an agent to bind to neovascular tissue. Some examples of agents that are used for this purpose are known to those of ordinary skill in the art for use in performing various tumor measurements, including measurement of the size of a tumor vascular bed, and measurement of tumor volume. Some of these agents bind to the vascular wall. One of ordinary skill in the art would be familiar with the agents that are available for use for this purpose. A tumor angiogenesis targeting ligand is a ligand that is used for the purpose of tumor angiogenesis targeting as defined above. Examples of angiogenesis targeting ligands include COX-2 inhibitors, anti-EGF receptor ligands, herceptin, angiostatin, C225 and thalidomide. COX-2 inhibitors include, for example, celecoxib, rofecoxib, etoricoxib and analogs of these agents.

3. Cardiac Ischemia Markers

The radionuclide-labeled chelator-glucose analog conjugates set forth herein may further be conjugated to a cardiac ischemia marker. Cardiac ischemia markers are discussed in U.S. patent application Ser. No. 11/770,395, herein incorporated by reference in its entirety. A cardiac ischemia marker is a ligand that is relatively selective for ischemic cardiac tissue. Non-limiting examples of cardiac ischemia markers include interleukin-6, tumor necrosis factor alpha, matrix metalloproteinase 9, myeloperoxidase, intercellular and vascular adhesion molecules, soluble CD40 ligand, placenta growth factor, high sensitivity C-reactive protein (hs-CRP), ischemia modified albumin (IMA), free fatty acids, and choline.

4. Viable Cardiac Tissue Markers

The radionuclide-labeled chelator-glucose analog conjugates set forth herein may further be conjugated to a viable cardiac tissue marker. Viable cardiac tissue markers are discussed in U.S. patent application Ser. No. 11/770,395, herein incorporated by reference in its entirety. A viable cardiac tissue marker refers to a ligand that is relatively selective for viable cardiac tissue compared to nonviable cardiac tissue. Non-limiting examples of cardiac viable tissue markers include those selected from the group consisting of phospholipase C, myosin light-chain phosphatase, nitric oxide, prostacyclin, endothelin, thromboxane, L-arginine and L-citrulline.

5. Congestive Heart Failure Markers

The radionuclide-labeled chelator-glucose analog conjugates set forth herein may further be conjugated to a congestive heart failure marker. Congestive heart failure markers are discussed in U.S. patent application Ser. No. 11/770,395, herein incorporated by reference in its entirety. A congestive heart failure marker is a ligand that is relatively selective for cardiac tissue of a heart in congestive heart failure compared to normal healthy heart tissue. Non-limiting examples of congestive heart failure markers include those selected from the group consisting of interleukin-1, cardiotrophin-1, insulin-like growth factor, epidermal growth factor, tyrosine kinase receptor and angiotensin II.

6. Rest/Stress Cardiac Tissue Markers

The radionuclide-labeled chelator-glucose analog conjugates set forth herein may further be conjugated to a rest/stress cardiac tissue marker. Rest/stress cardiac tissue markers are discussed in U.S. patent application Ser. No. 11/770,395, herein incorporated by reference in its entirety. A rest/stress cardiac tissue marker is a ligand that is relatively selective for cardiac tissue that is stressed compared to non-stressed (at rest) cardiac tissue, or vice versa. Non-limiting examples of rest/stress cardiac tissue markers include those selected from the group consisting of mitogen-activated protein kinase, cyclic adenosine monophosphate, phospholipase C, phosphatidylinositol bisphosphate, isositol trisphosphate, diacylglycerol and tyrosine kinases.

7. Peripheral Vascular Disease Tissue Markers

The radionuclide-labeled chelator-glucose analog conjugates set forth herein may further be conjugated to a Peripheral Vascular Disease (PVD) tissue marker. A PVD tissue marker is a ligand that is relatively selective for disorders which include venous valvular incompetence and venous hypertension, deep venous thrombosis, pulmonary embolism, post phlebitis syndrome, varicose veins, plaques, emboli, and other vascular disease. Non-limiting examples of PAD markers include those selected from the group consisting of diuretics, beta blockers, calcium blockers, converting enzyme inhibitors.

8. Imaging Moieties

The radionuclide-labeled chelator-glucose analog conjugates set forth herein may further be conjugated to an imaging moiety. Imaging moieties are discussed in U.S. patent application Ser. No. 11/770,395, herein incorporated by reference in its entirety. As defined herein, an “imaging moiety” is a part of a molecule that is an agent or compound that can be administered to a subject, contacted with a tissue, or applied to a cell for the purpose of facilitating visualization of particular characteristics or aspects of the subject, tissue, or cell through the use of an imaging modality. Imaging modalities are discussed in greater detail below. Any imaging agent known to those of ordinary skill in the art is contemplated as an imaging moiety of the present invention. Thus, for example, in certain embodiments of compositions of the present invention, the compositions can be applied in multimodality imaging techniques. Dual imaging and multimodality imaging are discussed in greater detail in the specification below.

In certain embodiments, the imaging moiety is a contrast media. Examples include CT contrast media, MM contrast media, optical contrast media, ultrasound contrast media, or any other contrast media to be used in any other form of imaging modality known to those of ordinary skill in the art. Examples include diatrizoate (a CT contrast agent), a gadolinium chelate (an MRI contrast agent) and sodium fluorescein (an optical contrast media). Additional examples of contrast media are discussed in greater detail in the specification below. One of ordinary skill in the art would be familiar with the wide range of types of imaging agents that can be employed as imaging moieties in the chelators of the present invention.

### E. Synthesis of Radionuclide-Labeled Chelator-Glucose Analogs

Reagents for preparation of compositions disclosed herein can be obtained from any source. A wide range of sources are known to those of ordinary skill in the art. For example, the reagents can be obtained from commercial sources such as Sigma-Aldrich Chemical Company (Miwaukee, Wis.), from chemical synthesis, or from natural sources. For example, one vendor of radionuclides is Cambridge Isotope Laboratories (Andover, Mass.). The reagents may be isolated and purified using any technique known to those of ordinary skill in the art, as described herein. The free unbound metal ions can be removed with, for example, ion-exchange resin or by adding a transchelator (e.g., glucoheptonate, gluconate, glucarate, or acetylacetonate).

1. Obtaining a Chelator

Methods of preparing and obtaining chelators are well known to those of skill in the art. For example, chelators may be obtained from commercial sources, chemical synthesis, or natural sources.

In some embodiments, the chelator is ethylenedicysteine (EC). The preparation of ethylenedicysteine (EC) is described in U.S. Pat. No. 6,692,724, herein incorporated by reference. Briefly, EC may be prepared in a two-step synthesis according to the previously described methods (Ratner and Clarke, 1937; Blondeau et al., 1967; each incorporated herein by reference). The precursor, L-thiazolidine-4-carboxylic acid, was synthesized and then EC was then prepared. It is often also important to include an antioxidant in the composition to prevent oxidation of the ethylenedicysteine. The preferred antioxidant for use in conjunction with the present invention is vitamin C (ascorbic acid). However, it is contemplated that other antioxidants, such as tocopherol, pyridoxine, thiamine, or rutin may also be useful.

Chelators may also comprise amino acids joined together by spacers. Such a spacer may comprise, as described above, an alkyl spacer such as ethylene.

Amide bonds may also join one or more amino acids together to form a chelator. Examples of synthetic methods for the preparation of such chelators include solid-phase synthesis and solution-phase synthesis. Such methods are described, for example, in Bodansky, 1993 and Grant, 1992.

2. Conjugation of Chelator to Glucose Analog and Other Ligands

Conjugation of a chelator to a glucose analog or other ligand may be by any method known to those of ordinary skill in the art. In some embodiments, conjugation takes place in an aqueous medium. Information concerning conjugation of a chelator to a ligand (e.g., glucose analog or other ligand) in an aqueous medium can be found, for example, in U.S. Pat. Nos. 7,223,380, 7,582,281, and U.S. patent application Ser. No. 11/405,334, herein incorporated by reference. In some embodiments, the chelator is dissolved in a basic aqueous solution and coupling agents of any type are added. Coupling agents, as used herein, are reagents used to facilitate the coupling of a chelator to a targeting ligand. Such agents are well known to those of ordinary skill in the art and may be employed in certain embodiments of methods of the present invention. Examples of coupling agents include, but are not limited to, sulfo-N-hydroxysuccinimide (sulfo-NHS), dimethylaminopyridine (DMAP), diazabicyclo[5.4.0]undec-7-ene (DBU), 1-ethyl-3-(3-dimethylaminopropyl) carbodiimide (EDAC) and dicyclohexylcarbodiimide (DCC). Other carbodiimides are also envisioned as coupling agents. Coupling agents are discussed, for example, in Bodansky, 1993 and Grant, 1992. These coupling agents may be used singly or in combination with each other or other agents to facilitate conjugation. The glucose analog or other ligand is then added to this solution to generate the conjugate.

In other embodiments, conjugation takes place in an organic medium. Information concerning conjugation of a ligand to a chelator in an organic medium can be found in U.S. patent application Ser. No. 11/770,395, herein specifically incorporated by reference in its entirety. The method may include obtaining, for example, a chelator such as ethylenedicysteine (EC) as described above and admixing the EC with a thiol protecting group in an organic medium in order to protect both free thiols, resulting in an S—S′-bis-protected-EC, which is then admixed with an amino protecting group in an organic/aqueous medium in order to protect both free amines, resulting in an S—S′-bis-protected-N,N′-bis-protected-EC. This protected EC is then conjugated to a ligand of any type described herein via any mode of conjugation described herein followed by removal of the thiol and amino protecting groups, which results in a conjugate as set forth herein.

In some embodiments, conjugation between a chelator and a glucose analog or other ligand takes place in one step. In particular embodiments, the conjugation comprises a covalent attachment of a chelator to a targeting ligand, wherein the covalent attachment occurs in one step. Such one-step procedures are preferable as they minimize time, reagents, waste and loss of product.

One of ordinary skill in the art will be familiar with the means of conjugating ligands to various functional groups. Most commonly, as between the chelator and the ligand, one acts as the nucleophile and one acts as the electrophile such that conjugation takes place via a covalent bond. Non-limiting examples of such covalent bonds include an amide bond, an ester bond, a thioester bond and a carbon-carbon bond.

In general, the ligands for use in conjunction with the present invention will possess functional groups that are able to conjugate to one or more functional groups of a chelator, such as EC. For example, a ligand (e.g., glucose analog or other ligand) may possess a halogenated position that will react with a free amine of a chelator to form the conjugate. If functional groups are not available, or if an optimal functional group is not available, a desired ligand may still be conjugated to a chelator, such as EC, by adding a linker, such as ethylenediamine, amino propanol, diethylenetriamine, aspartic acid, polyaspartic acid, glutamic acid, polyglutamic acid, cysteine, glycine or lysine. For example, U.S. Pat. No. 6,737,247 discloses several linkers which may be used with the present invention. U.S. Pat. No. 5,605,672 discloses several “preferred backbones” which may be used as linkers in the present invention and is hereby incorporated by reference in its entirety. In certain embodiments, the chelator may be conjugated to a linker, and the linker is conjugated to a ligand. In other embodiments more than one linker may be used; for example, a chelator may be conjugated to a linker, and the linker is conjugated to a second linker, wherein the second linker is conjugated a ligand. In certain embodiments, two, three, four, or more linkers that are conjugated together may be used to conjugate a chelator and ligand. However, it is generally preferable to only use a single linker to conjugate a chelator and a ligand.

TABLE 1
Linkers

| Drug Functional | | |
|---|---|---|
| Group | Linker | Example |
| Aliphatic or phenolic- | EC-poly(glutamic acid) | estradiol, |
| OH | (MW 750-15,000) or | topotecan, |
| | EC poly(aspartic acid) | paclitaxel, |
| | (MW 2000-15,000) or | raloxifen |
| | bromo ethylacetate or | etoposide |
| | EC-glutamic acid or | |
| | EC-aspartic acid. | |
| Aliphatic or aromatic- | EC-poly(glutamic acid) | doxorubicin, |
| NH₂ or peptide | (MW 750-15,000) or | mitomycin C, |
| | EC-poly(aspartic acid) | endostatin, |
| | (MW 2000-15,000) or | annexin V, |
| | EC-glutamic acid | LHRH, octreotide, |
| | (mono- or diester) or | VIP |
| | EC-aspartic acid. | |
| Carboxylic acid or | Ethylene diamine, | methotrexate, folic |
| peptide | lysine | acid |

3. Chelation of Chelator-Glucose Analog Conjugates to a Radionuclide

Chelator—glucose analog conjugates may be chelated to a radionuclide of any type described herein. Such methods of chelation are well known to those of ordinary skill in the art and are described herein. Examples of methods of chelation of metal ions to chelator—targeting ligand conjugates are described, for example, in U.S. Pat. No. 6,692,724. Methods described herein where a metal ion is chelated to a chelator may also serve as examples of how to chelate a metal ion to a chelator—glucose analog conjugate.

Chelation of a Chelator-Glucose Analog Conjugate to a Radionuclide May Take Place in an aqueous medium or an organic medium. U.S. patent application Ser. No. 11/770,395, provides information concerning radionuclide labeling in an organic medium. Benefits of synthesizing labeled conjugates using organic synthesis include, for example, obtaining conjugates of high purity.

In certain embodiments, the chelator and the ligand may each contribute to the chelation of the metal ion. In preferred embodiments, the metal ion is chelated only to the chelator. The chelated metal ion may be bound via, for example, an ionic bond, a covalent bond, or a coordinate covalent bond (also called a dative bond). Methods of such coordination are well known to those of ordinary skill in the art. In one embodiment, coordination may occur by admixing a metal ion into a solution containing a chelator. In another embodiment, coordination may occur by admixing a metal ion into a solution containing a chelator-glucose analog conjugate. In one embodiment, chelation occurs to the chelator via an N2S2 chelate formed by the chelator, such as ethylenedicysteine (EC). The chelator and the ligand may each be protected by one or more protecting groups before or after chelation with the metal ion.

Chelation may occur at any atom or functional group of a chelator or ligand that is available for chelation. The chelation may occur, for example, at one or more N, S, O or P atoms. Non-limiting examples of chelation groups include NS₂, N₂S, S₄, N₂S₂, N₃S and NS₃, and O₄. In preferred embodiments, a metal ion is chelated to three or four atoms. In some embodiments, the chelation occurs among one or more thiol, amine or carboxylic acid functional groups. The chelation, in particular embodiments, may be to a carboxyl moiety of glutamate, aspartate, an analog of glutamate, or an analog of aspartate. These embodiments may include multiple metal ions chelated to poly(glutamate) or poly(aspartate) chelators. In some embodiments, chelation of the metal ion is to a glucose analog, such as to carboxyl groups. In preferred embodiments, the chelation is between one or more thiol groups and one or more amine groups of the chelator.

In some non-limiting examples, the radionuclide may be technetium, indium, rhenium, gallium, copper, holmium, platinum, gadolinium, lutecium, yttrium, cobalt, calcium, arsenic, or any isotope thereof. Any metal ion described herein may be chelated to a compound of the present invention.

4. Reducing Agents

For purposes of the present invention, when the radionuclide is technetium it is preferred that the Tc be in the +4 oxidation state. The preferred reducing agent for use this purpose is stannous ion in the form of stannous chloride (SnCl2) to reduce the Tc to its +4 oxidation state. However, it is contemplated that other reducing agents, such as dithionate ion or ferrous ion may be useful in conjunction with the present invention. It is also contemplated that the reducing agent may be a solid phase reducing agent.

### F. Imaging

1. Imaging Modalities

A variety of imaging modalities are complated for application in the methods of the present invention. Examples are set forth below.

a. Gamma Camera Imaging

A variety of nuclear medicine techniques for imaging are known to those of ordinary skill in the art. Any of these techniques can be applied in the context of the imaging methods of the present invention to measure a signal from the reporter. For example, gamma camera imaging is contemplated as a method of imaging that can be utilized for measuring a signal derived from the radionuclide-labeled chelator-glucose analog conjugates.

b. PET and SPECT

Radionuclide imaging modalities (positron emission tomography, (PET); single photon emission computed tomography (SPECT)) are diagnostic cross-sectional imaging techniques that map the location and concentration of radionuclide-labeled radiotracers. Although CT and Mill provide considerable anatomic information about the location and the extent of tumors, these imaging modalities cannot adequately differentiate invasive lesions from edema, radiation necrosis, grading or gliosis. PET and SPECT agents can be used to localize and characterize tumors by measuring metabolic activity.

PET and SPECT provide information pertaining to information at the cellular level, such as cellular viability. In PET, a patient ingests or is injected with a slightly radioactive substance that emits positrons, which interact with electrons to create an annihilation event that results in the emission of two 511 KeV photons at an angle of 180 degrees relative to each other. The PET device monitors these emissions as the substance moves through the body. In one common application, for instance, patients are given glucose with positron emitters attached, and their brains are monitored as they perform various tasks. Since the brain uses glucose as it works, a PET image shows where brain activity is high.

Closely related to PET is single-photon emission computed tomography, or SPECT. The major difference between the two is that instead of a positron-emitting substance which results in the emission of two photons in opposite directions, SPECT uses a radioactive tracer that emit a single low energy photon in one direction. SPECT is valuable for diagnosing coronary artery disease, and already some 11 million SPECT heart studies are done in the United States each year.

PET radiopharmaceuticals for imaging are commonly labeled with positron-emitters such as ¹¹C, ¹³N, ¹⁵O, ¹⁸F, ⁸²Rb, ⁶²Cu, and ⁶⁸Ga. Importantly, the chelator-glucose analog conjugate described herein can be radiolabeled with ⁶⁸Ga, thus providing an agent that can be used to diagnose ischemic disease using PET imaging technology.

SPECT radiopharmaceuticals are commonly labeled with gamma ray emitters such as ^{99m}Tc, ¹¹¹In and ¹²³I. Regarding brain imaging, PET and SPECT radiopharmaceuticals are classified according to blood-brain-barrier permeability (BBB), cerebral perfusion and metabolism receptor-binding, and antigen-antibody binding (Saha et al., 1994). The blood-brain-barrier SPECT agents, such as ^{99m}TcO4-DTPA, ²⁰¹Tl, and [⁶⁷Ga]citrate are excluded by normal brain cells, but enter into tumor cells because of altered BBB. SPECT perfusion agents such as [¹²³I]IMP, [^{99m}Tc]HMPAO, [^{99m}Tc]ECD are lipophilic agents, and therefore diffuse into the normal brain. Important receptor-binding SPECT radiopharmaceuticals include [¹²³I]QNE, [¹²³I]IBZM, and [¹²³I]iomazenil. These tracers bind to specific receptors, and are of importance in the evaluation of receptor-related diseases.

c. Computerized Tomography (CT)

Computerized tomography (CT) is contemplated as an imaging modality in the context of the present invention. By taking a series of X-rays, sometimes more than a thousand, from various angles and then combining them with a computer, CT make it possible to build up a three-dimensional image of any part of the body. A computer is programmed to display two-dimensional slices from any angle and at any depth.

In CT, intravenous injection of a radiopaque contrast agent can assist in the identification and delineation of soft tissue masses when initial CT scans are not diagnostic. Similarly, contrast agents aid in assessing the vascularity of a soft tissue or bone lesion. For example, the use of contrast agents may aid the delineation of the relationship of a tumor and adjacent vascular structures.

CT contrast agents include, for example, iodinated contrast media. Examples of these agents include iothalamate, iohexol, diatrizoate, iopamidol, ethiodol and iopanoate. Gadolinium agents have also been reported to be of use as a CT contrast agent (see, e.g., Henson et al., 2004). For example, gadopentate agents has been used as a CT contrast agent (discussed in Strunk and Schild, 2004).

d. Magnetic Resonance Imaging (MRI)

Magnetic resonance imaging (MRI) is an imaging modality that is newer than CT that uses a high-strength magnet and radio-frequency signals to produce images. The most abundant molecular species in biological tissues is water. It is the quantum mechanical “spin” of the water proton nuclei that ultimately gives rise to the signal in imaging experiments. In MRI, the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to code spatial information into the recorded signals. By collecting and analyzing these signals, it is possible to compute a three-dimensional image which, like a CT image, is normally displayed in two-dimensional slices.

Contrast agents used in MR imaging differ from those used in other imaging techniques. Their purpose is to aid in distinguishing between tissue components with identical signal characteristics and to shorten the relaxation times (which will produce a stronger signal on T1-weighted spin-echo MR images and a less intense signal on T2-weighted images). Examples of MRI contrast agents include gadolinium chelates, manganese chelates, chromium chelates, and iron particles.

Both CT and MRI provide anatomical information that aid in distinguishing tissue boundaries and vascular structure. Compared to CT, the disadvantages of MRI include lower patient tolerance, contraindications in pacemakers and certain other implanted metallic devices, and artifacts related to multiple causes, not the least of which is motion (Alberico et al., 2004). CT, on the other hand, is fast, well tolerated, and readily available but has lower contrast resolution than MRI and requires iodinated contrast and ionizing radiation (Alberico et al., 2004). A disadvantage of both CT and MRI is that neither imaging modality provides functional information at the cellular level. For example, neither modality provides information regarding cellular viability.

2. Dual Imaging

Certain embodiments of the present invention pertain to methods of imaging a site within a subject using two imaging modalities that involve measuring a first signal from a first source and a second signal from a second source. In one non-limiting example, the first source is a radionuclide-labeled chelator-glucose analog conjugate as set forth herein, and the second source is Th-201.

a. Dual Isotope Imaging

In some embodiments, two separate compounds are administered, each of which can be applied in performing imaging. For example, the first compound may be a radionuclide-labeled chelator-glucose analog conjugate and the second agent may be another imaging agent having a different energy associated with it's gamma ray emission. In a non-limiting example, the second imaging agent is T1-201. Imaging to detect a radionuclide-labeled chelator-glucose analog conjugate may precede, be concurrent with, or follow imaging to detect a second agent. The second agent may be an agent to localize in normal heart tissue and provide anatomical localization information to identify the specific portion of the heart which is shown to be ischemic by the radiolabeled chelator-glucose agent. Administration of the radionuclide-labeled chelator-glucose analog may precede, be concurrent with, or follow administration of the second imaging agent.

In some embodiments, the same imaging device is used to perform imaging of both the radionuclide-labeled chelator-glucose analog conjugate and the second agent. In other embodiments, different imaging devices are employed. One of ordinary skill in the art would be familiar with the imaging devices that are available for performance of a first imaging modality and a second imaging modality, and the skilled artisan would be familiar with use of these devices to generate images.

b. Single Agent Imaging

In some embodiments, the chelator (or glucose analog) is further conjugated to an imaging moiety as discussed above. A first signal is derived from the radionuclide-labeled chelator targeting ligand conjugate and a second signal is derived from the imaging moiety. As set forth above, any imaging modality known to those of ordinary skill in the art can be applied in these embodiments of the present imaging methods.

Imaging is performed at any time during or after administration of the imaging agent. For example, the imaging studies may be performed during or after administration of the conjugate. In some embodiments, the first imaging study is performed 1 sec, 1 hour, 1 day, or any longer period of time following administration of the conjugate, or at any time in between any of these stated times. In other embodiments the imaging procedure is dynamic, accumulating imaging information continuously from the time of injection to about 1 to about 3 hours post injection. The imaging information may be used to assess the uptake characteristics of the agent under various conditions of ischemia. The uptake, retention and washout characteristics of the agent in the ischemic region of the heart may be used to provide prognostic information on the seriousness of the ischemia and may also provide guidance on the most efficacious therapy for the specific patient being imaged. Therapy options such as revascularization versus medical management may be defined from the dynamic imaging information.

The second imaging study may be performed concurrently with the first imaging study, or at any time following the first imaging study. For example, the second imaging study may be performed about 1 sec, about 1 hour, about 1 day, or any longer period of time following completion of the first imaging study, or at any time in between any of these stated times. In certain embodiments of the present invention, the first and second imaging studies are performed concurrently such that they begin at the same time following administration of the agent. One of ordinary skill in the art would be familiar with performance of the various imaging modalities contemplated by the present invention.

### G. Radiolabeled Agents

A sufficient amount of radionuclide label must be administered to facilitate imaging. For example, in forming ^{99m}Tc radioactive complexes, it is generally preferred to form radioactive complexes in solutions containing radioactivity at concentrations of from about 0.01 millicurie (mCi) to about 300 mCi per mL.

Radiolabeled imaging agents provided by the present invention can be used for visualizing cardiovascular tissue in a mammalian body. In accordance with this invention, the imaging agents are administered by any method known to those of ordinary skill in the art. For example, administration may be in a single unit injectable dose. Any of the common carriers known to those with skill in the art, such as sterile saline solution or plasma, may be utilized after radiolabeling for preparing the compounds of the present invention for injection. Generally, a unit dose to be administered has a radioactivity of about 0.01 mCi to about 300 mCi, preferably about 5 mCi to about 10 mCi. The solution to be injected at unit dosage is from about 0.01 mL to about 10 mL.

After intravenous administration of a diagnostically effective amount of a composition of the present invention, imaging can be performed. Imaging of a site within a subject, such as an organ or tumor can take place, if desired, in hours or even longer, after the radio-labeled reagent is introduced into a patient. In most instances, a sufficient amount of the administered dose will accumulate in the area to be imaged within about 0.5 of an hour. As set forth above, imaging may be performed using any method known to those of ordinary skill in the art. Examples include PET, SPECT, and two types of gamma scintigraphy. In gamma scintigraphy, the radiolabel is a gamma-radiation emitting radionuclide and the radiotracer is located using a gamma-radiation detecting camera (PET or SPECT). The imaged site is detectable because the radiotracer is chosen either to localize at a pathological site (termed positive contrast).

### H. Methods of Diagnosis of Cardiovascular Disease and Peripheral Vascular Disease

Some embodiments of the present invention generally pertain to methods of diagnosing a subject with known or suspected cardiovascular disease (or peripheral vascular disease) or evaluating a subject with cardiovascular disease (or peripheral vascular disease) for disease progression. The subject can be any subject, such as a mammal or avian species. The mammal, for example, may be a dog, cat, rat, mouse, or human. In preferred embodiments, the subject is a human with known or suspected cardiovascular disease.

The cardiovascular disease can be any disease of the heart or of a blood vessel. The blood vessel may be a coronary vessel, or may be a vessel other than a coronary vessel. The vessel may be an artery, vein, arteriole, venule, or capillary.

Examples of cardiovascular diseases include diseases of the heart, such as myocardial infarction, myocardial ischemia, angina pectoris, congestive heart failure, cardiomyopathy (congenital or acquired), arrhythmia, or valvular heart disease. In particular embodiments, the subject is known or suspected to have myocardial ischemia.

The subject, for example, may be a patient who presents to a clinic with signs or symptoms suggestive of myocardial ischemia or myocardial infarction. Imaging of the heart of the subject to diagnose disease may involve administering to the subject a pharmaceutically effective amount of a metal ion labeled chelator-targeting ligand conjugate synthesized using any of the methods set forth herein. Imaging can be performed using any imaging modality known to those of ordinary skill in the art. In particular embodiments, imaging involves use radionuclide-based imaging technology, such as PET or SPECT. In particular embodiments, the metal ion-labeled radionuclide-targeting ligand conjugate is 99m-Tc-EC-glucosamine. Glucosamine is actively taken up by ischemic tissue. Areas of normal myocardium would take up less or no conjugate. Severity of ischemia can be visually assessed or graded depending on magnitude of the signal that is measured using any method known to those of ordinary skill in the art. In some embodiments, imaging using any of the conjugates set forth herein is performed before, during, or after imaging of the heart using a second imaging modality. For example, the second imaging modality may be thallium scintigraphy. Myocardial Perfusion SPECT (MPS) consists of a combination of a stress modality (exercise or pharmacologic) with rest and stress administration and imaging of radiopharmaceuticals. Thallium has excellent physiologic properties for myocardial perfusion imaging. Being highly extracted during the first pass through the coronary circulation, a linear relationship between blood flow to viable myocardium and thallium uptake has been shown during exercise; however, at very high levels of flow, a “roll-off”in uptake occurs. As an unbound potassium analogue, thallium redistributes over time. Its initial distribution is proportional to regional myocardial perfusion and at equilibrium, the distribution of thallium is proportional to the regional potassium pool, reflecting viable myocardium. The mechanisms of thallium redistribution are differential washout rates between hypoperfused but viable myocardium and normal zones and wash-in to initially hypoperfused zones. The washout rate of thallium is the concentration gradient between the myocardial cell and the blood. There is slower blood clearance of thallium following resting or low-level exercise injection. Diffuse slow washout rates, mimicking diffuse ischemia, may be observed in normal patients who do not achieve adequate levels of stress. Hyperinsulinemic states slow redistribution, leading to an underestimation of viable myocardium; thus fasting is recommended prior to and for 4 hrs following thallium injection. This is why if EC-G is used as a viability agent in combination with thallium it will target the precise area of interest which would be the viable area (Angello et al., 1987; Gutman et al., 1983; Pohost et al., 1977).

Examples of peripheral vascular disease include an obstruction to blood flow (such as a thrombus, plaque, emboli, etc.) in a blood vessel of a subject that is not in the heart. Examples of such blood vessels include femoral arteries, brachial arteries, subclavian arteries, carotid arteries, aorta, pulmonary artery, cerebral arteries, and veins and vessels of the bowel and brain.

Imaging using any of the metal ion-labeled chelator-targeting ligand conjugates of the present invention may also be performed in conjunction with other diagnostic methods, such as measurement of cardiac isozymes, or cardiac catheterization. The imaging may be performed at various intervals following onset of symptoms, or can be performed to assess for changes in myocardial perfusion over time.

### I. Kits

Certain embodiments of the present invention are generally concerned with kits for diagnostic imaging of a site of cardiovascular disease in a subject. For example, in some embodiments the kit includes one or more sealed containers that contain a predetermined quantity of a chelator-glucose analog conjugate and one or more sealed containers that contain a predetermined quantity of a second imaging agent that can be applied in imaging the heart of a subject. In some embodiments, the kit further includes a sealed containiner containing a radionuclide.

A kit of the present invention may further include a predetermined quantity of a chelator-glucose analog conjugate of the present invention and a sufficient amount of reducing agent to label the compound with a metal ion.

The kit may also contain conventional pharmaceutical adjunct materials such as, for example, pharmaceutically acceptable salts to adjust the osmotic pressure, buffers, preservatives and the like.

In certain embodiments, an antioxidant is included in the composition to prevent oxidation of the chelator moiety. In certain embodiments, the antioxidant is vitamin C (ascorbic acid). However, it is contemplated that any other antioxidant known to those of ordinary skill in the art, such as tocopherol, pyridoxine, thiamine, or rutin, may also be used. The components of the kit may be in liquid, frozen, dry form, or lyophilized form.

### J. Pharmaceutical Preparations

Pharmaceutical compositions of the present invention comprise a diagnostically and/or therapeutically effective amount of a radionuclide-labeled chelator-glucose analog conjugate as set forth herein. The phrases “pharmaceutical or pharmacologically acceptable” or “therapeutically effective” or “diagnostically effective” refers to molecular entities and compositions that do not produce an adverse, allergic or other untoward reaction when administered to an animal, such as, for example, a human, as appropriate. The preparation of therapeutically effective or diagnostically effective compositions will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by the FDA Office of Biological Standards.

As used herein, “a composition comprising a diagnostically effective amount” or “a composition comprising a therapeutically effective amount” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with the active ingredient, its use in the present compositions is contemplated.

The compositions of the present invention may comprise different types of carriers depending on whether it is to be administered in solid, liquid or aerosol form, and whether it need to be sterile for such routes of administration as injection. The compositions of the present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, injection, infusion, continuous infusion, localized perfusion bathing target cells directly, via a catheter, via a lavage, in lipid compositions (e.g., liposomes), or by any other method or any combination of the forgoing as would be known to one of ordinary skill in the art.

The actual required amount of a composition of the present invention administered to a patient can be determined by physical and physiological factors such as body weight, severity of condition, the tissue to be imaged, the type of disease being treated, previous or concurrent imaging or therapeutic interventions, idiopathy of the patient, and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of the chelator—metal ion chelate. In other embodiments, the an active compound may comprise between about 2% to about 75% of the weight of the unit, or between about 25% to about 60%, for example, and any range derivable therein. In other non-limiting examples, a dose may also comprise from about 0.1 mg/kg/body weight to about 1000 mg/kg/body weight or any amount within this range, or any amount greater than 1000 mg/kg/body weight per administration.

In any case, the composition may comprise various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof

The compositions of the present invention may be formulated in a free base, neutral or salt form. Pharmaceutically acceptable salts include the salts formed with the free carboxyl groups derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

Sterile injectable solutions may be prepared using techniques such as filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO (dimethylsulfoxide) as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### K. Examples

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

The following figures, chemical structures and synthetic details provide certain compounds of the present invention.

### Example 1

### Use of Tc-99m-Labeled Glucosamine Analog for the Noninvasive Assessment of Myocardial Injury Following Acute Myocardial Infarction

The purpose of this study was to evaluate 99mTc-ethylenedicysteine (EC) glucosamine in cardiovascular models of disease, particularly in the setting of post infarction left ventricular remodeling where the myocardial tissue is undergoing marked changes in associate with the reparative processing following a severe injury.

### Results

Heart Failure Resulting from Rapid Right Atrial Pacing.

In two dogs with right atrial pacing at 180 beats per minute (bpm) for 5 weeks, there was significant deterioration of cardiac ejection fraction at the 5 week timepoint with ejection fraction reduced to less than 25% in both cases. Magnetic resonance (MR) imaging at 5 weeks showed concentric dilatation of the left ventricular (LV) chamber. The dogs underwent 99mTc-EC-glucosamine SPECT using a LEHR collimator, 360 degree rotation, 64 stops, at 30 seconds/stop. Upon completion of the 99mTc-EC-glucosamine SPECT, the dogs were injected with about 40 mCi of 99mTc-tetrofosmin and a repeat SPECT image of perfusion was obtained.

The uptake of tetrofosmin in the LV of one dog was not completely homogenous, most likely as a result of microinfarction and/or interstitial fibrosis consequent to LV remodeling. Likewise, 99mTc-EC-glucosamine uptake in the same region was patchy with relatively low activity levels throughout the LV.

In the other dog, similar results were obtained in the LV, with patchy 99mTc-EC-glucosamine uptake seen in the LV, particularly in the septal wall. However, there was also a significant amount of blood pool activity even at one hour post injection. It is believed that the high activity in the blood pool resulted from poor labeling efficiency of the 99mTc-EC-glucosamine tracer.

Heart Failure Resulting from Myocardial Infarction.

In this model, one of the major coronary arteries, either the left anterior descending (LAD) or left circumflex (LCx) coronary artery and all visible collateral vessels are tied off for 120 minutes, followed by complete reflow of the vessels. The chest is closed and the dogs are recovered and followed for up to 8 weeks with MR and SPECT imaging. In some dogs, dual isotope SPECT using 99mTc-EC-glucosamine followed by tetrofosmin perfusion imaging was performed, whereas in other experiments, 99mTc-EC-glucosamine uptake is compared with thallium-201 (T1201) distribution using ex vivo imaging of heart slices and gamma well counting. Autoradiography and gamma well counting of 99mTc activity is not possible when using a 99mTc perfusion agent at the same time as 99mTc-EC-glucosamine.

In vivo MR and ex vivo autoradiography of the heart at eight weeks post myocardial infarction was evaluated in one dog. Hyperenhanced regions of the LV using Gd-DTPA, an MR contrast agent that depicts areas of necrosis, were identified. Focal hot spots of 99mTc-EC-glucosamine uptake were identified in ex vivo images of 99mTc-EC-glucosamine uptake in the same heart slices imaged directly on the gamma camera collimator. The same areas that can take up 99mTc-EC-glucosamine are those showing injury by cardiac MR.

Likewise, tetrofosmin staining of the heart slices showed similar patterns of injury to the papillary muscle and mid-ventricular region of the LV.

The heart slices were then cut into segments and counted for both T1-201 and 99mTc-EC-glucosamine uptake. FIG. 1 shows that there was enhanced uptake of 99mTc-EC-glucosamine relative to perfusion.

In another dog, 99mTc-EC-glucosamine and tetrofosmin imaging was performed at baseline and at 48 hours post infarction. There was no 99mTc-EC-glucosamine uptake observed in the myocardium, with some uptake seen in the blood pool at one hour post injection. At 48 hours post myocardial infarction, there was a small focal hot spot of 99mTc-EC-glucosamine in the apex of the heart. Corresponding to a relatively small apical infarct in this dog. At 8 weeks, ex vivo imaging of heart slices showed some enhancement of 99mTc-EC-glucosamine in the more apical two slides of the LV.

In another dog, 99mTc-EC-glucosamine uptake was compared with T1-201 perfusion by ex vivo imaging at 8 weeks post infarction. MRI performed at 48 hours and 8 weeks showed a large anteroseptal region of myocardial injury in this dog. There was focal uptake of 99mTc-EC-glucosamine corresponding to the Tl201 defect areas that represents significant injury and myocardial necrosis.

Based on the results obtained, it was decided to modify the protocol to allow more time (2 hours) between the injection of the 99mTc-EC-glucosamine tracer and the imaging, to perform the first image at 72 hours rather than 48 hours, and to split the final study at 8 weeks to allow both in vivo SPECT and ex vivo imaging. Final SPECT images of 99mTc-EC-glucosamine and tetrofosmin will be performed at 7.5 weeks followed by 99mTc-EC-glucosamine and thallium ex vivo imaging at 8 weeks. Baseline images of the dog showed high blood pool activity as was observed in earlier in the RV pacing dog. It is possible that the improved labeling efficiency in the 72 hour and 1 week scans resulted from changes that were made in the instructions for reconsistituting the 99mTc-EC-glucosamine kits. Despite the high blood pool activity, no 99mTc-EC-glucosamine was observed in the myocardium. At 72 hours, there was a large amount of 99mTc-EC-glucosamine uptake observed in the posterior wall corresponding to a posterior wall hyperenhancement on cardiac MR. This particular dog had a LCx occlusion that produced an infarct size measuring nearly 50% of the LV. At one week, 99mTc-EC-glucosamine uptake was still detectable in the same region, although the overall amount had diminished relative to the 72 hour time point. This dog will undergo further imaging at 4 weeks and again at 7.5/8 weeks as described above.

Ischemic Memory—Acute Model.

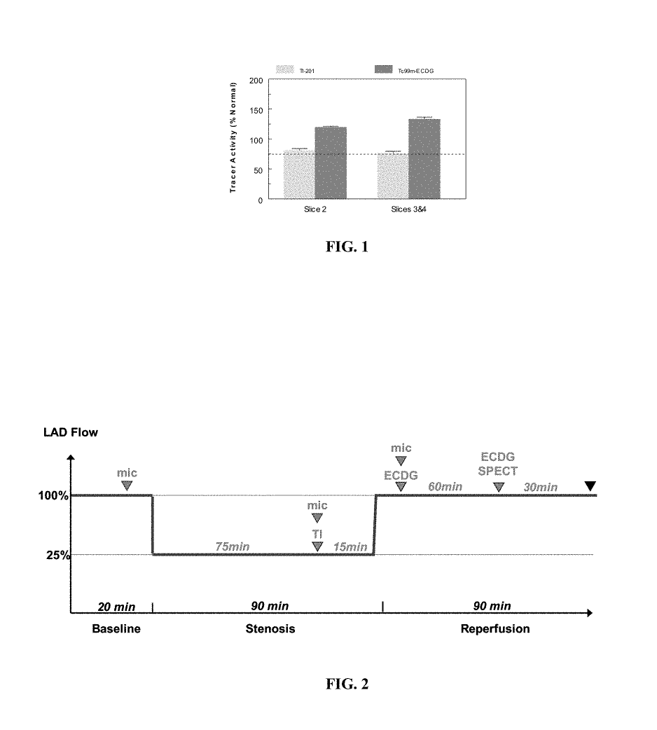

In two dogs, LAD flow was reduced by 75% by tightening a snare occluder to produce resting ischemia for 90 minutes followed by full reperfusion. 99mTc-EC-glucosamine was injected 15 minutes after reflow with SPECT imaging performed at one hour post injection. No perfusion images were acquired in these studies, however, the perfusion image shown from another dog in the same position on the table is fused with the 99mTc-EC-glucosamine images for orientation purposes only (FIG. 2).

In the first dog, in vivo SPECT imaging showed a focal 99mTc-EC-glucosamine hot spot in the apex of the heart. Likewise, ex vivo imaging of heart slices showed a similar hotspot in the most apical slide of the heart.

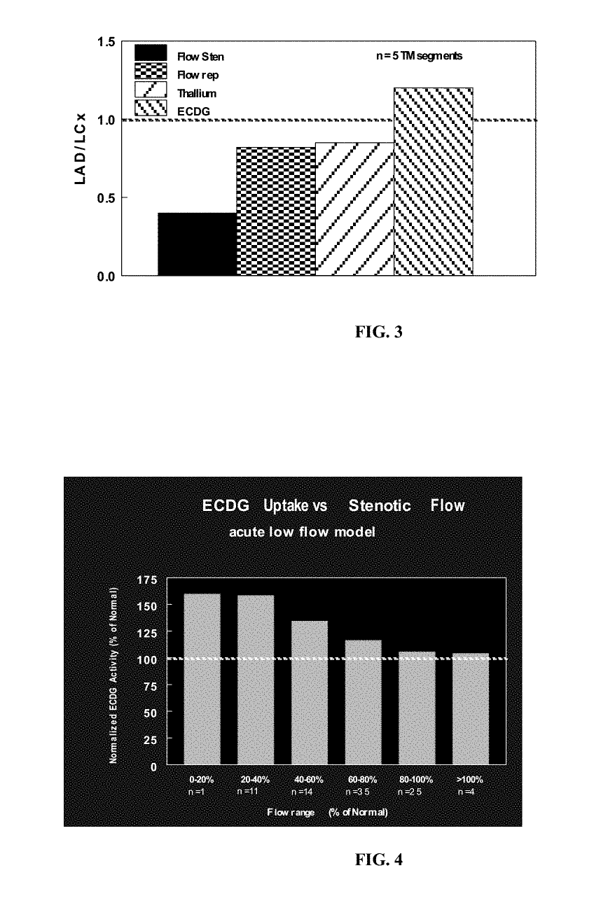

The heart slides were divided into 96 segments and counted in a gamma well counter for microsphere flow, Tl-201 activity, and 99mTc-EC-glucosamine uptake. As shown by microspheres, flow in the LAD zone was reduced by more than 50% relative to the normal zone during the ischemic phase. After reperfusion, both flow and Tl-201 activity in the LAD zone were slightly reduced relative to the normal zone, but 99mTc-EC-glucosamine activity in the LAD zone was higher than in the normal LCx zone (FIG. 3).

When subdivided into categories according to the degree of flow reduction during ischemia, it is apparent that 99mTc-EC-glucosamine uptake is enhanced in the central and border zone ischemic areas. Even in mildly ischemic areas where flow was reduced by only 20-40% of normal, there was enhanced 99mTc-EC-glucosamine uptake. In the central ischemic zone where flow was reduced by 60-80% of normal, 99mTc-EC-glucosamine uptake was highest with greater than 150% activity relative to the normal zone (FIG. 4).

In the second dog, similar results were obtained with a focal hotspot of 99mTc-EC-glucosamine seen in the apex of the heart.

Ischemic Memory—Chronic Model.

In a third dog, the protocol above was modified to examine 99mTc-EC-glucosamine uptake at later time points after reperfusion. In addition, the severity of the ischemic insult was increased such that LAD flow was reduced by 90% rather than 75% for 90 minutes. 99mTc-EC-glucosamine uptake was assessed at 48 hours and one week after reperfusion. Tetrofosmin staining at the end of the study revealed no necrosi in the myocardium. Even with a relatively mild ischemic insult lasting 90 minutes without infarction, 99mTc-EC-glucosamine uptake was observed 48 hours later. However, unlike the more severe injury of myocardial infarction in the studies described above, no uptake of 99mTc-EC-glucosamine was observed at one week. These results indicate that the duration of a positive 99mTc-EC-glucosamine signal after ischemic injury may be dependent on the severity of the injury.

The results show that 99mTc-EC-glucosamine is taken up in the region of myocardial injury and can be imaged by in vivo SPECT as late as one week after myocardial infarction in the case of a more severe injury. Uptake of 99mTc-EC-glucosamine can be found even at 8 weeks after infarction by ex vivo autoradiography.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,605,672
U.S. Pat. No. 6,692,724
U.S. Pat. No. 6,737,247
U.S. Pat. No. 7,067,111
U.S. Pat. No. 7,223,380
U.S. Pat. No. 7,229,604
U.S. Pat. No. 7,582,281
U.S. application Ser. No. 09/599,152
U.S. application Ser. No. 10/627,763
U.S. application Ser. No. 10/672,142
U.S. application Ser. No. 10/703,405
U.S. application Ser. No. 10/732,919
U.S. application Ser. No. 11/405,334
U.S. application Ser. No. 11/770,395
U.S. Patent Publn. 20040166058
U.S. Patent Publn. 20050129619
U.S. Patent Publn. 20070248537
U.S. Patent Publn. 20080107598
Alberico et al., Surg. Oncol. Clin. N. Am., 13(1):13-35, 2004.
Angello et al., Am. J. Cardiol., 60:528-533, 1987.
Becker et al., J. Am. Coll. Cardiol., 14:1491-1500, 1989.
Benveniste and Davies, Proc. Natl. Acad. Sci. USA, 70(8):2276-2280, 1973.
Blondeau et al., Can. J. Chem., 45:49-52, 1967.
Bodansky, In: Peptide Chemistry, 2^{nd} ed., Springer-Verlag, New York, 1993.
Borodina et al., Appl. Environ. Microb., 71(5):2294-302, 2005.
Diamond et al., J. Biol. Chem., 253(3):866-871, 1978.
Grant, In: Synthetic Peptides, Freeman & Co., New York, 1992.
Gutman et al., Am. Heart J., 106: 989-995, 1983.
Hendel et al., J Nucl Cardiol., 3:291-300, 1996.
Henson et al., AMR Am. J. Neuroradiol., 25(6):969-972, 2004.
Hoelscher et al., Spine, 25(15):1871-7, 2000.
Hostetler and Hall, Proc. Natl. Acad. Sci. USA, 79:1663-1667, 1982.
Hu, Proc. Natl. Acad. Sci USA, 95; 9791-95, 1998.
Ilgan et al., Cancer Biother. Radiopharm., 13(6):427-35, 1998.
Michalik et al., Pharmacol Res., 21(4):405-414, 1989.
Murakami et al., Bone, 21(5):411-418, 1997.
Murakami et al., J. Orthop. Res., 14(5):742-8, 1996.
Myszka et al., Carb. Res., 338:133-141, 2003.
Nakae and Nakae, Antimicrobial Agents and Chemo., Oct; 22(4):554-59, 1982.
Niess et al., Circulation, 59:1010-1019, 1979.
Ozmen et al., Drug Chem. Toxicol., 28(4):433-45, 2005.
Pohost et al., Circulation, 55:294-302, 1977.
Ratner and Clarke, J. Am. Chem. Soc., 59:200-206, 1937.
Remington's Pharmaceutical Sciences, 18th Ed. Mack Printing Company, 1289-1329, 1990.
Saha et al., Semin. Nucl. Med., 24(4):324-49, 1994.
Strunk and Schild, Eur. Radiol., 14(6):1055-1062, 2004.
Tachibana et al., Biochem. Pharmacol., 25(20):2297-301, 1976.
Tod et al., Clin Pharmacokinet., 38(3):205-223, 2000.
Wackers et al., NEJM, 295(1):1-5, 1976.
Yanai et al., Proc. Natl. Acad. Sci. USA, 103(25):9661-9666, 2006.
Yang et al., Ann. Nucl. Med., 20(a):1-11, 2006.
Yang et al., Diabetes, 53:67-73, 2004.
Zareneyrizi et al., Anticancer Drugs, 10(7):685-92, 1999.

1. A method of imaging a site in a heart of a subject to detect cardiovascular disease, the method comprising detecting a signal generated by a radionuclide-labeled chelator-glucose analog conjugate in the heart of the subject, wherein a site of ischemia in the heart, if present, generates a signal that is more intense than surrounding heart tissue.

2-58. (canceled)

59. A method of imaging a site in a heart of a subject to detect cardiovascular disease, wherein the subject has been previously subjected to stress and imaged using nuclear imaging to determine whether there is a region of decreased perfusion suggesting the presence of ischemia, the method comprising:
(a) administering to the patient at rest a detectable amount of a radionuclide-labeled chelator-glucose analog conjugate; and
(b) imaging the heart of the subject to detect a signal generated by the radionuclide-labeled chelator-glucose analog conjugate,
wherein a signal generated by the radionuclide-labeled chelator-glucose analog conjugate in the region of the heart that showed decreased perfusion on the MPI imaging study is a region of suspected myocardial ischemia.

60-63. (canceled)

64. A method of distinguishing a false positive nuclear cardiology scan from a true positive nuclear cardiology scan, comprising:
a) administering to a subject a detectable amount of a radionuclide-labeled chelator-glucose analog conjugate, wherein the subject has had a positive nuclear cardiology scan, and a site of diminished signal was identified on the nuclear cardiology scan that is suggestive of cardiac ischemia; and
(b) imaging the heart of the subject to detect the presence of a signal generated by the radionuclide-labeled chelator-glucose analog conjugate,
wherein the presence of a signal that is more intense than surrounding heart tissue is indicative of a true positive nuclear cardiology scan, and absence of a signal that is more intense than surrounding heart tissue is indicative of a false positive nuclear cardiology scan.

65-87. (canceled)

88. A method to diagnose congestive heart failure or monitor response to treatment of congestive heart failure in a subject, the method comprising detecting a signal generated by a radionuclide-labeled chelator-glucose analog conjugate in the heart of the subject, wherein a site of ischemia in the heart, if present, generates a signal that is more intense than surrounding heart tissue and is indicative of the presence of cardiac ischemia.

89-104. (canceled)

105. A method to diagnose a previous episode of cardiac ischemia in a subject that is suspected of having had a previous episode of cardiac ischemia, the method comprising detecting a signal generated by a radionuclide-labeled chelator-glucose analog conjugate in the heart of the subject, wherein a site of ischemia in the heart, if present, generates a signal that is more intense than surrounding heart tissue and is indicative of the presence of past or current cardiac ischemia.

106-124. (canceled)
